(19) United States
(12) Patent Application Publication
(10) Pub. No.: US 2025/0229020 A1
(43) Pub. Date: Jul. 17, 2025

(54) SYSTEM AND METHOD FOR MONITORING ACCURACY OF A SYRINGE PUMP

(71) Applicant: CareFusion 303, Inc., San Diego, CA (US)

(72) Inventor: Hermogenes ESCALA, Limerick City (IE)

(21) Appl. No.: 18/692,787

(22) PCT Filed: Mar. 14, 2023
(86) PCT No.: PCT/US2023/015221

§ 371 Date: Mar. 15, 2024

(51) Int. Cl. A61M 5/168; A61M 5/145

(52) U.S. Cl. CPC A61M 5/1684; A61M 5/14546; A61M 2005/16863; A61M 2205/18; A61M 2205/3306; A61M 2205/3327; A61M 2205/3365

(57) ABSTRACT

The subject technology provides for monitoring accuracy of an infusion device, particularly a syringe pump. A drive head pushes on a plunger to infuse a fluid from a syringe. During the infusion, a location of a drive head of the device is monitored using one or more proximity sensors. The monitored location is compared to a location determined by way of mechanical means and if different by more than a threshold amount, a safety operation is initiated, which may include an alert, change in motor speed, or termination of the infusion.

### BACKGROUND

This application relates generally to ensuring that a syringe pump is functioning as expected.

The infusion of medical fluids, such as parenteral fluids, into the human body is accomplished in many cases by means of a syringe pump in which a syringe containing the parenteral fluid is mounted. Syringe pumps typically secure the syringe barrel in a fixed position and push or “drive” the syringe plunger into the barrel at a controlled rate to expel the parenteral fluid. A fluid administration set conducts the expelled parenteral fluid from the syringe barrel to the patient. Many syringe pumps have an elongated lead screw rotated by a motor and a screw drive mechanism such as a split nut that translates the rotational motion of the lead screw into linear motion. A syringe plunger driver is connected to the screw drive mechanism and to the syringe plunger for driving the plunger into the syringe barrel in accordance with the movement of the lead screw to expel the parenteral fluid.

Accuracy is important when delivering medication to patient through an infusion device such as a syringe pump. Manufacturers determine accuracy under nominal conditions and operations, for example, at ambient temperature, nominal infusion rate, and using a common size of syringe. As these factors vary, the infusion accuracy can deviate from the nominal conditions and operations. Some infusion devices rely on the motor and motor encoder to approximate the volume infused or remaining volume-to-be-infused (VTBI) during an ongoing infusion. While the motor drives the lead screw, all turns of the motor may not directly relate to a fluid expulsion. For example, a worn-out lead screw may go undetected for a period of time. In such instances, the motor encoder may treat each cycle as if the lead screw was in proper working order. However, due to deterioration of the lead screw, a motor cycle may cause the lead screw to travel more or less than if in proper working order. This can lead to inaccuracies in determining the amount fluid expelled by the device based on motor encoding (e.g., over-infusion or under-infusion).

### SUMMARY

The subject technology monitors accuracy of an infusion device. In this regard, the subject technology includes an infusion system, comprising: a receptacle for receiving a syringe, the syringe comprising a barrel and a plunger; a motor-operated drive head for advancing the plunger within the barrel during an infusion; a first sensor for determining a location of the drive head based on operation of the motor during the infusion; a second sensor for determining the location of the drive head independently of the first sensor and the motor; a controller configured to: operate the motor to advance the plunger within the barrel; obtain, using the first sensor, a first measurement of the location of the drive head based on operation of the motor during the infusion; obtain, using the second sensor contemporaneously with obtaining the first measurement, a second measurement of the location of the drive head; determine whether the second measurement deviates from the first measurement by more than a first threshold deviation; and provide a first alert when the second measurement deviates from the first measurement by more than the first threshold deviation.

In some implementations, the infusion system comprises: a third sensor for measuring the location of the drive head from a side of the drive head opposite the second sensor, wherein the controller is further configured to: obtain, using the third sensor contemporaneously with obtaining the second measurement, a third measurement of the location of the drive head; determine whether the third measurement deviates from the second measurement by more than a second threshold deviation; and provide a second alert when the second measurement deviates from the first measurement by more than the second threshold deviation. Other aspects include corresponding apparatuses, methods, and computer program products for implementation of the corresponding infusion system and its features.

The subject technology also relates to a method for monitoring accuracy of a syringe pump. In this regard, the method comprises: receiving an indication that a syringe was loaded into a receptacle of an infusion device, the syringe comprising a barrel and a plunger; advancing, during an infusion, a motor-operated drive head of the infusion device to advance the plunger within the barrel; obtaining, using a first sensor, a first measurement of location of the drive head based on operation of the motor during the infusion; obtaining, using a second sensor contemporaneously with obtaining the first measurement, a second measurement of the location of the drive head; determining whether the second measurement deviates from the first measurement by more than a first threshold deviation; and providing a first alert when the second measurement deviates from the first measurement by more than the first threshold deviation. Other aspects include corresponding systems, apparatus, and computer program products for implementation of the corresponding method and its features.

It is understood that other configurations of the subject technology will become readily apparent to those skilled in the art from the following detailed description, wherein various configurations of the subject technology are shown and described by way of illustration. As will be realized, the subject technology is capable of other and different configurations and its several details are capable of modification in various other respects, all without departing from the scope of the subject technology. Accordingly, the drawings and detailed description are to be regarded as illustrative in nature and not as restrictive.

### BRIEF DESCRIPTION OF THE DRAWINGS

For a better understanding of the various described implementations, reference should be made to the Description below, in conjunction with the following drawings. Like reference numerals refer to corresponding parts throughout the figures and description.

FIG. 1 depicts an example syringe infusion pump, according to aspects of the subject technology.

FIG. 2 depicts the example syringe pump control system, including a proximity sensor for monitoring accuracy of the syringe pump's drive head, according to various aspects of the subject technology.

FIG. 3 depicts an example process for monitoring accuracy of a syringe pump, according to aspects of the subject technology.

FIG. 4 is a conceptual diagram illustrating an example electronic system for monitoring accuracy of a syringe pump, according to aspects of the subject technology.

### DESCRIPTION

Reference will now be made to implementations, examples of which are illustrated in the accompanying drawings. In the following description, numerous specific details are set forth, in order to provide an understanding of the various described implementations. However, it will be apparent to one of ordinary skill in the art that the various described implementations may be practiced without these specific details. In other instances, well-known methods, procedures, components, circuits, and networks have not been described in detail so as not to unnecessarily obscure aspects of the implementations.

The subject technology provides for monitoring accuracy of an infusion device, particularly a syringe pump. A drive head pushes on a plunger to infuse a fluid from a syringe. During the infusion, a location of a drive head is monitored using one or more proximity sensors. The monitored location is compared to a location determined by way of mechanical means (e.g., a conventional encoding mechanism) and, if different by more than a threshold amount, a safety operation is initiated which may include an alert, change in motor speed, or termination of the infusion.

The proximity sensors detect actual movement of the drive head, as opposed to existing technologies that primarily rely on an encoded representation of the lead screw. Using the proximity sensors of the subject technology, the disclosed system may obtain real-time measurement and feedback for the volume of fluid infused by way of accurate detection of the drive head position. In this regard, the volume infused (or flow rate) may be accurately estimated based on the location of the drive head, as measured by the proximity sensors.

According to various implementations, the sensors determine the distance of the actuator on each side and provide that information to the pump controller continuously during an infusion. The infusion device compares the measurements to calculated volume and may adjust the motor speed or recalculate the volume based on the proximity measurements. In this manner, accuracy of the infusion device is improved.

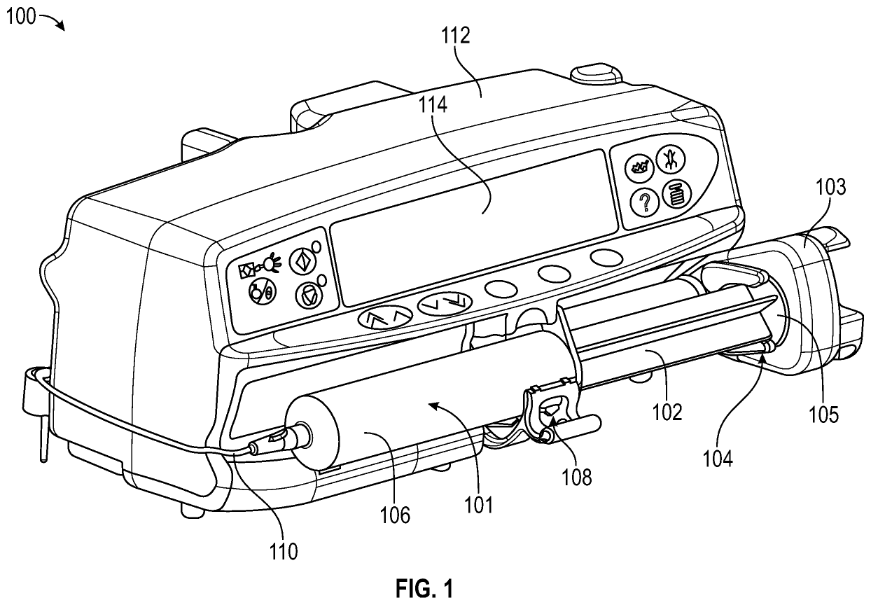

FIG. 1 depicts an example syringe pump 100 infusion device, according to various aspects of the subject technology. While the example syringe pump 100 is shown as a stand alone device, the syringe pump may be configured as a functional module of a modular infusion system such as a syringe module for the BD Alaris™ system.

When a syringe 101 is loaded in the syringe pump 100, a plunger flange (or push button) 105 at the end of a syringe plunger piston 102 is held in or against a plunger drive head 103 by a flange clamp 104. The syringe barrel 106 is secured by a syringe clamp 108. The drive head 103 includes a pushing surface on which the plunger flange 105 will rest as the drive head 103 moves forward toward the syringe barrel 106 pushing the plunger piston 102 into the barrel 106 of the syringe to expel the syringe contents through an administration tubing 110 to the patient. As will be described further, the drive head 103 may be connected to a screw drive mechanism, including a motor, for connecting the linear motion of the screw drive mechanism to the syringe plunger in order to empty the syringe. The rate is controlled by the syringe pump 100 based on programmed parameters (e.g., desired rate, type of syringe).

Syringe pumps do not typically experience any upstream pressure conditions because the fluid to be infused is housed in the syringe barrel 106 and is pushed into an administration set 110 by way of the plunger piston 102. Downstream pressure conditions can be detected by a force sensor housed in or upon a pump system 112. In some implementations, a force sensor measures the force exerted by the drive head 103 of the syringe pump on the syringe plunger piston 102.

In some implementations, the syringe pump 100 may include a high-resolution pressure sensor that interfaces with a pressure disc (not shown) on the syringe administration set. The pressure disc provides a relatively large area in contact with the pressure sensor. This allows the pressure sensor to measure the pressure inside the administration set more directly (not through the syringe plunger head) and with higher resolution and higher accuracy compared with the drive head force sensor. The measurements from this pressure sensor and the drive head force sensor can be used independently or in conjunction with each other to detect an empty condition in a syringe pump.

As well as operating buttons or switches, which the operator may use to activate and program the syringe pump 100, there is a display screen 114. The display screen 114 may be an LCD (liquid crystal display) having a small number of segments, for example seven segments in a figure-of-eight configuration per character, adapted to display a small number of alphanumeric characters. The display may be monochromatic, for example, it might only display red, green, or grey/black characters. Alternatively, the display 114 can be a more complicated liquid crystal display capable of displaying more characters or more complicated characters. The LCD may be backlit, for example, using light emitting diodes (LEDs). In some implementations, the infusion pump may include a TFT LCD. A TFT is a thin-film transistor-based LCD technology. In some implementations, the display screen 114 is also a touchscreen such as a capacitive touchscreen.

When programming the syringe pump 100, the user may input the type of syringe being used. The syringe pump 100 may store in an internal memory a database of known syringe types containing information such as syringe diameter and stroke. The infusion pump firmware calculates the position of the syringe plunger and syringe piston based on movement of the syringe drive head and the type and size of the syringe. This allows the machine to display the calculation of volume infused, time elapsed, volume remaining and time remaining. As infusion continues and the drive head moves, these calculations can be updated, and the displayed information changed.

The syringe pump 100 may be provided with an input interface with controls operable to enter, increase or decrease pumping parameters, such as the mass flow rate setting shown on a display, or the VTBI (volume to be infused) setting shown on the display. In some cases, an input key may be physically present on the device (as depicted) or may be graphically displayed in a touchscreen display 114.

In some implementations, the syringe pump 100 may be configured to identify (e.g., using a sensor) a disposable container loaded by the device. For example, a syringe pump 100 may perform electro-mechanical measurements on the loaded syringe to identify certain characteristics about the loaded container. For example, a syringe pump may include a sensor that measures the size of the syringe inserted into the pump, for example, based on how tightly the syringe is being hugged. The clock position may determine the size of the barrel (e.g., whether it is a 6, 10, 50 ml syringe, etc.). Based on the physical measurements made by the pump, the syringe pump may determine a list of possible candidate syringes. The device may then request confirmation via the display whether the container is within that list. During the infusion, volume infused and/or flow rate may be calculated based on the syringe type (e.g., based on the size of the syringe barrel).

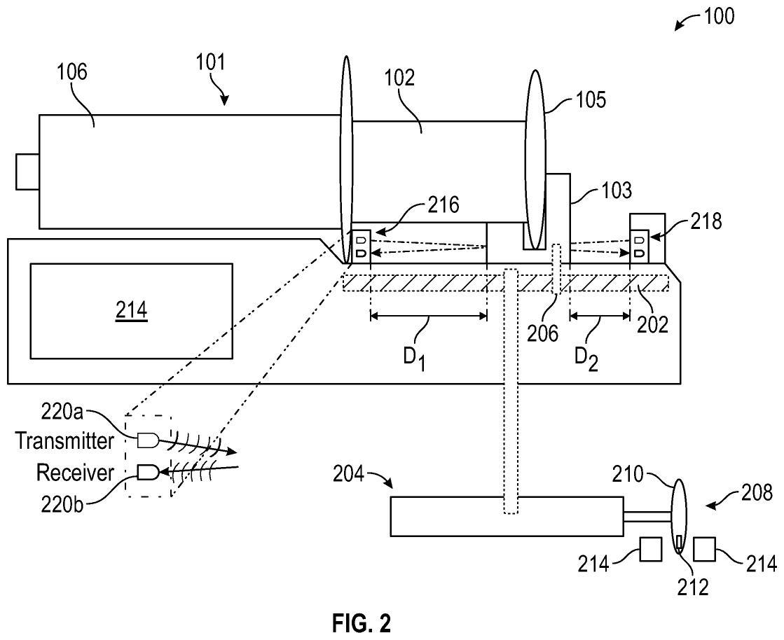

FIG. 2 depicts the example syringe pump control system, including a proximity sensor for monitoring accuracy of the syringe pump's drive head, according to various aspects of the subject technology. According to various implementations, the syringe pump includes components for controlling advancement of the plunger piston 102 within the barrel 106 of a syringe 101. The drive head 103, which engages the push-button 105 of the syringe 101, can be moved by a drivetrain, including a lead screw 202. For example, the syringe pump 100 can include a motor assembly 204, including a motor that operates to rotate the lead screw 202. The lead screw 202 may be engaged by a screw drive mechanism 206 (not shown in detail), such as a split nut, that translates the rotational motion of the lead screw 202 into linear motion. The drive head 103 is connected to the screw drive mechanism 206 and to the plunger 102 for driving the plunger piston 102 into the barrel 106 in accordance with the movement of the lead screw 202 to expel fluid from the barrel 106.

According to some embodiments, the motor assembly 204 can include a stepper motor, a brushed DC electric motor, a brushless DC electric motor, a servo motor, an AC motor, or another type of motor. The drivetrain of the motor assembly can include appropriate gears, axles, shafts, chains, hydraulics, and/or any other components for translating rotational motion of the motor into linear motion of the drive head 103. According to some embodiments, the drivetrain, including the motor, can include linear actuating components, such as a linear stepper motor. For example, a linear motor can act directly on the drive head 103, or a component attached thereto, to control linear motion thereof.

According to various implementations, the motor assembly 204, cam, and/or lead screw 202 of the syringe pump 100 may be coupled to an encoder device 208. In this manner, the encoder device 208 may encode the turns of the motor, cam, or lead screw for use by the pump's processor in determining a distance the drive head has traveled and/or its current location. The encoder device 208 may include an encoder wheel 210 that includes one or more holes 212 that rotate past a receiver-transmitter pair 214. A continuous light may be transmitted by the transmitter of the receiver-transmitter pair 214. As the encoder wheel 210 rotates, the receiver of the receiver-transmitter pair 214 may detect the light through the hole(s) and the encoder device 208 may determine the distance that the drive head 103 has traveled based on the number of rotations (or partial rotations) of the encoder wheel 210.

The encoder device 208 may transmit a signal to the infusion pump's controller 215 which may determine an amount or volume of fluid infused. According to various implementations, the encoder device 208 can provide an absolute measurement on the plunger itself. In some instances, its resolution accuracy may be checked and corrected, if necessary, thus giving more reliable information.

In some infusion operations, an end-user such as a clinician can select a flow rate and a VTBI (volume to be infused). The flow rate is typically in milliliters/hour and the VTBI is typically in milliliters. By using these two known inputs, the pump processor unit 215 can calculate the total duration that it must operate to complete the infusion using, for example, Equation 1.

$$Duration = \frac{VTBI}{Flow Rate} \qquad ( Eq . 1 )$$

To calculate if the required Duration of the first equation is already met, the pump controller 215 may obtain feedback from an encoder wheel 210 which is attached to the pumping motor. The number of encoder-per-revolution of the encoder wheel 210 is known. A motor speed will also be pre-determined at a given flow rate. Therefore, if the pump completes a number of encoders at the pre-determined motor speed, it estimates that the Duration is already completed. And by virtue of Eq. 1, the VTBI is complete:

$$Duration = Number of Encoders / ( Motor Speed \times Encoders per Revolution ) \qquad ( Eq . 2 )$$

Accordingly, the volume infused may be estimated based on the equation:

$$Volume Infused = ( Flow Rate ) \times ( Number of Encoders ) / \qquad ( Eq . 3 )$$

$$( Motor Speed \times Encoders per Revolution )$$

The encoder device 208 may function as a first sensor configured to measure a current location or distance travelled by the drive head 103 based on operation of the motor assembly 204. According to various implementations, the syringe pump 100 also includes a second sensor 216 and (optionally) a third sensor 218 positioned on opposite sides of the drive head 103. According to various implementations, each of the first and second sensors is an optical transceiver pair 220. An optical transceiver pair 220 is arranged on or in the syringe pump 100 to emit an optical signal toward a portion of the drive head 103 and to measure at least a portion of the optical signal reflected from the portion of the drive head 103.

In some implementations, the second and/or third sensor may include an infrared (IR) proximity sensor(s) 216, 218 configured to emit and receive infrared light. In some implementations, each proximity sensor 216, 218 may include one or more photodiodes. In the depicted example of sensor 216, a voltage is applied to a pair of IR photodiodes, including a transmitting photodiode 220 a and a receiving photodiode 220 b. Sensor 218 may implement the same configuration. If there is an object in front of the sensor 216 when the transmitter 220 a emits, some of the emission (e.g., light) is reflected and picked up by the receiver 220 b. The receiver 220b may be an IR photodiode that detects the reflected IR light and changes its conductivity based on the amount of IR light it receives. The conductivity may then be transposed into an equivalent distance, how far the objected detected from the sensor, which then can be translated into a location. The IR sensor can detect objects from short to long distance. Other forms of proximity sensors may be used.

As will be described further, the syringe pump utilizes at least two sensors for determining the location of the drive head 103. Accordingly, in some implementations, the syringe pump 100 includes proximity sensor 216 and proximity sensor 218. In some implementations, the syringe pump 100 includes encoder device 208 and proximity sensor 216. In some implementations, the syringe pump 100 includes encoder device 208 and proximity sensor 216 and proximity sensor 218.

In the depicted example, a syringe is shown received into a receptacle (e.g., secured by a syringe clamp 108) of syringe pump 100. During an infusion, the controller 215 operates the motor assembly 204 to advance the drive head 103 to advance the plunger 102 into the syringe barrel 106. The controller 215 obtains, using the first sensor (e.g., from the encoder device), a first measurement of a location of the drive head (e.g., distance travelled by the drive head) based on operation of the motor assembly. The controller 215 obtains, using the second sensor 216 contemporaneously with obtaining the first measurement (e.g., at the same time), a second measurement of the location of the drive head independent of the motor assembly. In the depicted example, a distance is measured by reflecting an optical signal off a bottom portion of the drive head/actuator, between the plunger and the syringe pump housing. In some implementations, at the same time, the controller 215 obtains third measurement of the location of the drive head using the third sensor 218. As depicted, the third sensor may be placed on the syringe pump housing behind the drive head 103 and may measure the distance from a side of the drive head 103 opposite the second sensor 218. For the purposes of this disclosure, the terms first and second sensor may be referred to interchangeably, irrespective of their respective locations.

According to various implementations, the controller 215 determines whether the second measurement deviates from the first measurement by more than a first threshold deviation. The controller 215 may also determine whether the third measurement deviates from the second measurement by more than a second threshold deviation. In some implementations, the second measurement represents a first distance D₁ between the second sensor 216 and a first side of the drive head 103, and the third measurement represents a second distance D₂ between the third sensor and a second side of the drive head, opposite the first side. In this regard, the controller 215 may continuously monitor the distances during an infusion and detect when the third measurement deviates from the second measurement by more than the second threshold deviation based on determining the first distance is different than the second distance by more than a threshold distance.

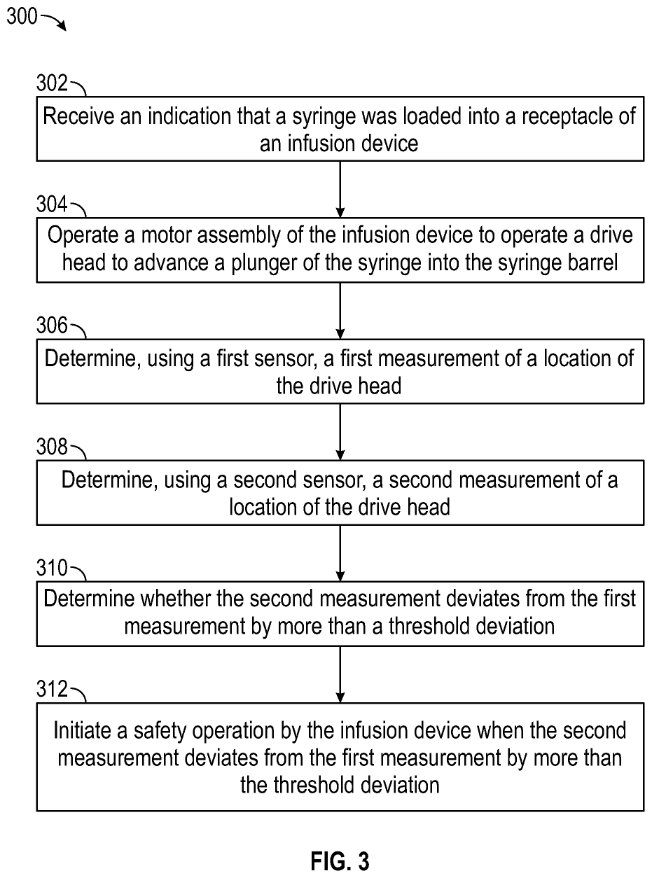

FIG. 3 depicts an example process 300 for monitoring accuracy of a syringe pump drive head, according to aspects of the subject technology. For explanatory purposes, the various blocks of example process 300 are described herein with reference to FIGS. 1 and 2, and the components and/or processes described herein. The one or more of the blocks of process 300 may be implemented, for example, by one or more computing devices including, for example, within an infusion device 100. In some implementations, one or more of the blocks may be implemented based on one or more machine learning algorithms. In some implementations, one or more of the blocks may be implemented apart from other blocks, and by one or more different processors or devices. Further for explanatory purposes, the blocks of example process 300 are described as occurring in serial, or linearly. However, multiple blocks of example process 300 may occur in parallel. In addition, the blocks of example process 300 need not be performed in the order shown and/or one or more of the blocks of example process 300 need not be performed.

In the depicted example, an indication is received by the infusion device 100 (e.g., by the controller 215) that a syringe 101 was loaded into a receptacle of an infusion device (302). As described previously, the infusion device 100 includes a drive head 103 that advances the plunger piston 102 into the syringe barrel 106 according to a programmed therapy. In some implementations, the type of syringe is detected by the syringe pump responsive to being loaded into the receptacle. Type of syringe may be received via user input or may be detected automatically by the infusion device 100 such as via a barrel sizer (not shown). The controller 215 may calculate a volume infused based on the syringe type (e.g., size) and the distance which the drive head 103 has moved.

When the infusion is initiated, a motor assembly 204 of the infusion device is operated (e.g., by the controller 215) to advance a drive head 103 and hence the plunger within the barrel 106 of the syringe (304).

The controller 215 determines (e.g., generates) and/or obtains, using a first sensor, a first measurement of a location of the drive head 103 (306). For example, the first sensor may provide a measurement indicating a distance travelled by the drive head, and the measurement may then be translated by the controller 215 to a respective location on the pump. According to some implementations, the first measurement is based on operation of the motor assembly. For example, the first sensor may include an encoder device 208 that determines the distance the drive head 103 has moved by way of monitoring the turns of the motor or lead screw 202.

The controller 215 determines (e.g., generates) and/or obtains, using a second sensor, contemporaneously with obtaining the first measurement, a second measurement of the location of the drive head independent of the motor assembly (308). For example, the second sensor may provide a measurement indicating a distance between the sensor and the drive head, and the measurement may then be translated by the controller 215 to a respective location on the pump. In some implementations, the second measurement is a distance (e.g., linear distance D₁) between the second sensor and a first side of the drive head.

In various implementations, the second sensor includes a proximity sensor. For example, the second sensor may include an optical transceiver pair, and the controller 215 may cause the the optical transceiver pair to emit an optical signal and measure a portion of the optical signal reflected from a portion of the drive head to measure the distance. While the second sensor is depicted and described as a proximity sensor and the first sensor as an encoder device, the encoder device may be omitted (or disabled) and both the first and second sensors are proximity sensors. The portion of the optical signal may be measured for quantity (e.g., how much of the emitted signal is reflected) or for time (e.g., how long it takes for a reflection to be detected by the receiver) or for a characteristic (e.g., color hue, intensity, brightness, etc. of emitted signal received). One or more of these measurements can be used to generate a distance of the drive head from the optical transceiver pair and estimate the location of the drive head.

The controller 215 determines/detects whether the second measurement deviates from the first measurement by more than a threshold deviation (310). In some implementations, the measurements are translated to locations (e.g., coordinate locations) and the locations compared, and the threshold deviation may be a threshold difference between locations (e.g., measured in distance). In some implementations, a distance is determined for the first sensor (e.g., based on encoder measurements) and compared to the distance determined by the second sensor. In some implementations, descriptions herein of distance and/or location may be replaced by volume infused. For example, a volume infused may be calculated for each measurement (e.g., based on distance, syringe type, etc.) and the volumes (e.g., from each sensor) compared to determine whether there is a difference in volumes greater than a predetermined threshold volume or other correspondence between one or more of: the calculated volumes or the predetermined threshold or value generated based thereupon. In some implementations the predetermined threshold, or other thresholds described herein, may be dynamically defined based on, for example, the type of syringe loaded in the infusion device or type of infusion device. In some implementations, a threshold discussed may be a static value (e.g., stored in memory of the infusion device).

Responsive to the determined deviation satisfying the threshold deviation, the controller may initiate a safety operation. In some implementations, the controller 215 provides a first alert when the second measurement deviates from the first measurement by more than the first threshold deviation (312). In some implementations, a speed of the motor assembly is adjusted responsive to the second measurement deviating from the first measurement by more than the first threshold deviation. In some implementations, the speed of the motor assembly is adjusted by adjusting the speed to zero to terminate operation of the motor assembly.

Before the motor is terminated, the controller 215 may take new measurements from one or more of the sensors to confirm the deviation satisfies the threshold deviation. For example, the controller may compare at least two new measurements obtained from using at least two of the first sensor, second sensor and third sensor. The controller 215 may compare a new measurement obtained from the first sensor with a new measurement obtained from the second and/or third sensor. The controller 215 may only compare a new measurement obtained from the second sensor with a new measurement obtained from the third sensor. On confirming the threshold deviation based on the new measurements, the infusion device operation may be performed.

In some implementations, a third measurement of the distance travelled by the drive head is obtained using a third sensor, contemporaneously with obtaining the first and/or second measurements. As depicted in FIG. 2, the third sensor may measure the distance travelled by the drive head from a side of the drive head opposite the second sensor. Accordingly, the controller 215 may determine whether the third measurement deviates from the second measurement by more than a second threshold deviation, and provide an alert when the second measurement deviates from the first measurement by more than the second threshold deviation.

In some implementations, responsive to the second measurement deviating from the first measurement by more than the first threshold deviation, the first sensor may be deactivated and the third sensor activated. In this manner, control of the infusion (e.g., measuring volume infused) may be entirely determined based on the distance measured by the first and second sensors.

In some implementations, the third measurement corresponds to a second distance D₂ between the third sensor and a second side of the drive head, opposite the first side. In this regard, the controller 215 may determine/detect (during an infusion) that the third measurement deviates from the second measurement by more than the second threshold deviation. For example, the distances D₁ and D₂ may be normalized and the threshold deviation may be satisfied based on determining the first (normalized) distance is different than the second distance by more than a threshold distance. In some implementations, determining the threshold deviation is satisfied includes the ratio between the distances being greater than a predetermined ratio or outside a ratio tolerance.

In some implementations, the alert may indicate a blockage in a path of the plunger or the drive head and prompt a user to confirm removal of the blockage before the motor assembly is restarted. In some implementations, the alert may indicate the blockage when the threshold deviation is satisfied for the proximity sensors (e.g., second and third sensors). In some implementations, the alert may indicate the blockage when the second sensor measurement does not correspond to the first sensor measurement.

According to various implementations, the controller 215 is configured to continuously determine a volume infused (or flow rate) based new measurements obtained from the first sensor (e.g., based on the encoder). In some implementations, when the proximity sensor(s) sensors that the location of the drive head 103 does not correspond to the location reported by the first sensor (e.g., by more than a threshold deviation) the controller 215 may deactivate or disable the first sensor for the purpose of determining the volume infused (or flow rate). The controller 215 may switch determining of the volume infused from being based on further/new measurements obtained from the first sensor to being based on new measurements obtained from the second sensor(s).

Many of the above-described example process 300, and related features and applications, may also be implemented as software processes that are specified as a set of specific instructions recorded on a computer readable storage medium (also referred to as computer readable medium), and may be executed automatically (e.g., without user intervention). When these specific instructions are executed by one or more processing unit(s) (e.g., one or more processors, cores of processors, or other processing units), they cause the processing unit(s) to perform the actions indicated in the instructions. Examples of computer readable media include, but are not limited to, CD-ROMs, flash drives, RAM chips, hard drives, EPROMs, etc. The computer readable media does not include carrier waves and electronic signals passing wirelessly or over wired connections.

The term “software” is meant to include, where appropriate, firmware residing in read-only memory or applications stored in magnetic storage, which can be read into memory for processing by a processor. Also, in some implementations, multiple software aspects of the subject disclosure can be implemented as sub-parts of a larger program while remaining distinct software aspects of the subject disclosure. In some implementations, multiple software aspects can also be implemented as separate programs. Finally, any combination of separate programs that together implement a software aspect described here is within the scope of the subject disclosure. In some implementations, the software programs, when installed to operate on one or more electronic systems, define one or more specific machine implementations that execute and perform the operations of the software programs.

A computer program (also known as a program, software, software application, script, or code) can be written in a programming language, including compiled or interpreted languages, declarative or procedural languages, and it can be deployed in one or more forms, including as a stand-alone program or as a module, component, subroutine, object, or other unit suitable for use in a computing environment. A computer program may, but need not, correspond to a file in a file system. A program can be stored in a portion of a file that holds other programs or data (e.g., one or more scripts stored in a markup language document), in a single file dedicated to the program in question, or in multiple coordinated files (e.g., files that store one or more modules, sub programs, or portions of code). A computer program can be deployed to be executed on one computer or on multiple computers that are located at one site or distributed across multiple sites and interconnected by a communication network.

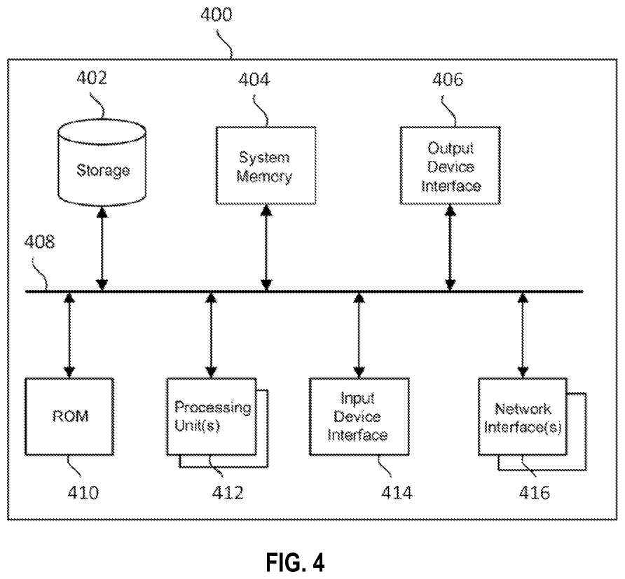

FIG. 4 is a conceptual diagram illustrating an example electronic system 400 for automatically programming a medical device, including bypassing portions of an infusion programming workflow, according to aspects of the subject technology. Electronic system 400 may be a computing device for execution of software associated with one or more portions or steps of process 400, or components and methods provided by FIGS. 1-3, including but not limited to computing hardware within the infusion device 100, and/or other specifically configured computing devices or associated terminals disclosed herein. In this regard, electronic system 400 may be a personal computer or a mobile device such as a smartphone, tablet computer, laptop, personal digital assistant (PDA), an augmented reality device, a wearable such as a watch or band or glasses, or combination thereof, or other touch screen or television with one or more processors embedded therein or coupled thereto, or other sort of computer-related electronic device having network connectivity specifically configured to implement one or more of the features described.

Electronic system 400 may include various types of computer readable media and interfaces for various other types of computer readable media. In the depicted example, electronic system 400 includes a bus 408, processing unit(s) 412, a system memory 404, a read-only memory (ROM) 410, a permanent storage device 402, an input device interface 414, an output device interface 406, and one or more network interfaces 416. In some implementations, electronic system 400 may include or be integrated with other computing devices or circuitry for operation of the various components and methods previously described.

Bus 408 collectively represents system, peripheral, resource, and chipset buses that communicatively connect the numerous internal devices of electronic system 400. For instance, bus 408 communicatively connects processing unit(s) 412 with ROM 410, system memory 404, and permanent storage device 402.

From these various memory units, processing unit(s) 412 retrieves instructions to execute and data to process, in order to execute the processes of the subject disclosure. The processing unit(s) can be a single processor or a multi-core processor in different implementations.

ROM 410 stores static data and instructions that are needed by processing unit(s) 412 and other modules of the electronic system. Permanent storage device 402, on the other hand, is a read-and-write memory device. This device is a non-volatile memory unit that stores instructions and data even when electronic system 400 is off. Some implementations of the subject disclosure use a mass-storage device (such as a magnetic or optical disk and its corresponding disk drive) as permanent storage device 402.

Other implementations use a removable storage device (such as a floppy disk, flash drive, and its corresponding disk drive) as permanent storage device 402. Like permanent storage device 402, system memory 404 is a read-and-write memory device. However, unlike storage device 402, system memory 404 is a volatile read-and-write memory, such as, random access memory. System memory 404 stores some of the instructions and data that the processor needs at runtime. In some implementations, the processes of the subject disclosure are stored in system memory 404, permanent storage device 402, and/or ROM 410. From these various memory units, processing unit(s) 412 retrieves specific instructions to execute and data to process, in order to execute the processes of some implementations.

Bus 408 also connects to input and output device interfaces 414 and 406. Input device interface 414 enables the user to communicate information and select commands to the electronic system. Input devices used with input device interface 414 include, e.g., alphanumeric keyboards and pointing devices (also called “cursor control devices”). Output device interfaces 406 enables, e.g., the display of images generated by the electronic system 400. Output devices used with output device interface 406 include, e.g., printers and display devices, such as cathode ray tubes (CRT) or liquid crystal displays (LCD). Some implementations include devices such as a touchscreen that functions as both input and output devices.

Also, as shown in FIG. 4, bus 408 also couples electronic system 400 to a network (not shown) through network interfaces 416. Network interfaces 416 may include, e.g., a wireless access point (e.g., Bluetooth or WiFi) or radio circuitry for connecting to a wireless access point. Network interfaces 416 may also include hardware (e.g., Ethernet hardware) for connecting the computer to a part of a network of computers such as a local area network (“LAN”), a wide area network (“WAN”), wireless LAN, a personal area network (“PAN”), or an Intranet, or a network of networks, such as the Internet. Any or all components of electronic system 400 can be used in conjunction with the subject disclosure.

These functions described above can be implemented in specifically configured computer software, firmware, or hardware. The techniques can be implemented using one or more computer program products. Programmable processors and computers can be included in or packaged as mobile devices. The processes and logic flows can be performed by one or more programmable processors and by one or more programmable logic circuitry. General and special purpose computing devices and storage devices can be interconnected through communication networks to cooperatively perform specific aspects of the described features.

Some implementations include electronic components, such as microprocessors, storage and memory that store computer program instructions in a machine-readable or computer-readable medium (also referred to as computer-readable storage media, machine-readable media, or machine-readable storage media). Some examples of such computer-readable media include RAM, ROM, read-only compact discs (CD-ROM), recordable compact discs (CD-R), rewritable compact discs (CD-RW), read-only digital versatile discs (e.g., DVD-ROM, dual-layer DVD-ROM), a variety of recordable/rewritable DVDs (e.g., DVD-RAM, DVD-RW, DVD+RW, etc.), flash memory (e.g., SD cards, mini-SD cards, micro-SD cards, etc.), magnetic and/or solid state hard drives, read-only and recordable Blu-Ray® discs, ultra density optical discs, any other optical or magnetic media, and floppy disks. The computer-readable media can store a computer program that is executable by at least one processing unit and includes sets of specific instructions for performing various operations described herein. Examples of computer programs or computer code include machine code, such as is produced by a compiler, and files including higher-level code that are executed by a computer, an electronic component, or a microprocessor using an interpreter.

While the above discussion primarily refers to specific microprocessor or multi-core processors that execute software, some implementations are performed by one or more integrated circuits, such as application specific integrated circuits (ASICs) or field programmable gate arrays (FPGAs) specifically configured with one or more of the features described. In some implementations, such integrated circuits execute instructions that are stored on the circuit itself.

As used in this specification and any claims of this application, the terms “computer”, “server”, “processor”, and “memory” all refer to electronic or other technological devices. These terms exclude people or groups of people. For the purposes of the specification, the terms display or displaying means displaying on an electronic device. As used in this specification and any claims of this application, the terms “computer readable medium” and “computer readable media” are entirely restricted to tangible, physical objects that store information in a form that is readable by a computer. These terms exclude any wireless signals, wired download signals, and any other ephemeral signals.

To provide for interaction with a user, implementations of the subject matter described in this specification can be implemented on a specifically configured computer having a display device, e.g., a CRT (cathode ray tube) or LCD (liquid crystal display) monitor, for displaying information to the user and a keyboard and a pointing device, e.g., a mouse or a trackball, by which the user can provide input to the computer. Other kinds of specifically configured devices can be used to provide for interaction with a user as well; e.g., feedback provided to the user can be any form of sensory feedback, e.g., visual feedback, auditory feedback, or tactile feedback; and input from the user can be received in any form, including acoustic, speech, or tactile input. In addition, a computer can interact with a user by sending documents to and receiving documents from a device that is used by the user; e.g., by sending web pages to a web browser on a user's client device in response to requests received from the web browser.

Implementations of the subject matter described in this specification can be implemented in a specifically configured computing system that includes a back end component, e.g., as a data server, or that includes a middleware component, e.g., an application server, or that includes a front end component, e.g., a client computer having a graphical user interface or a Web browser through which a user can interact with an implementation of the subject matter described in this specification, or any combination of one or more such back end, middleware, or front end components. The components of the system can be interconnected by any form or medium of digital data communication, e.g., a communication network. Examples of communication networks include a local area network (“LAN”) and a wide area network (“WAN”), an inter-network (e.g., the Internet), and peer-to-peer networks (e.g., ad hoc peer-to-peer networks).

The computing system can include clients and servers. A client and server are generally remote from each other (e.g., physically separated) and may interact through a communication network. The relationship of client and server arises by virtue of computer programs running on the respective computers and having a client-server relationship to each other. In some embodiments, a server transmits data (e.g., an HTML page) to a client device (e.g., for purposes of displaying data to and receiving user input from a user interacting with the client device). Data generated at the client device (e.g., a result of the user interaction) can be received from the client device at the server.

Although the discussion references the use of container information within an APR to improve the accuracy and safety of device configuration, the features may be implemented in other medical devices that currently include a container selection. For example, an intravenous compounding device may include selection of a container into which a medication will be compounded. The selection is typically performed manually or, in some instances, based on image recognition. Similar to the faults that can arise discussed above, manual entry or image recognition can misidentify containers. The misidentification may impact further processes of the compounding system such as identification of tare weight, container compatibility with drugs or components included in the preparation, volumetric verification of the preparation, and the like. Accordingly, such systems may receive container information as part of the order to compound a medication or other workflow including a container selection.

Those of skill in the art would appreciate that the various illustrative blocks, modules, elements, components, methods, and algorithms described herein may be implemented as electronic hardware, computer software, or combinations of both. To illustrate this interchangeability of hardware and software, various illustrative blocks, modules, elements, components, methods, and algorithms have been described above generally in terms of their functionality. Whether such functionality is implemented as hardware or software, depends upon the particular application, and design constraints imposed on the overall system. The described functionality may be implemented in varying ways for each particular application. Various components and blocks may be arranged differently (e.g., arranged in a different order, or partitioned in a different way) all without departing from the scope of the subject technology.

It is understood that the specific order or hierarchy of steps in the processes disclosed is an illustration of example approaches. Based upon design preferences, it is understood that the specific order or hierarchy of steps in the processes may be rearranged. Some of the steps may be performed simultaneously. The accompanying method claims present elements of the various steps in a sample order, and are not meant to be limited to the specific order or hierarchy presented.

### Illustration of Subject Technology as Clauses:

Various examples of aspects of the disclosure are described as numbered clauses (1, 2, 3, etc.) for convenience. These are provided as examples, and do not limit the subject technology. Identifications of the figures and reference numbers are provided below merely as examples and for illustrative purposes, and the clauses are not limited by those identification.

Clause 1. An infusion system comprising: a receptacle for receiving a syringe, the syringe comprising a barrel and a plunger; a motor-operated drive head for advancing the plunger within the barrel; a first sensor for determining a location of the drive head while the syringe is received in the receptacle; a second sensor for determining the location of the drive head independently of the first sensor and the motor; and a controller configured to: operate the motor to advance the plunger within the barrel; determine, using the first sensor, a first measurement of the location of the drive head while the syringe is received in the receptacle; determine, using the second sensor contemporaneously with obtaining the first measurement, a second measurement of the location of the drive head; determine that the second measurement deviates from the first measurement by a first threshold deviation; and provide a first alert after determining that the second measurement deviates from the first measurement by more than the first threshold deviation.

Clause 2. The infusion system of Clause 1, wherein the first measurement is determined based on operation of the motor, the system further comprising: a third sensor for measuring the location of the drive head from a side of the drive head opposite the second sensor, wherein the controller is further configured to: determine, using the third sensor contemporaneously with obtaining the second measurement, a third measurement of the location of the drive head; determine whether the third measurement deviates from the second measurement by more than a second threshold deviation; and provide a second alert when the second measurement deviates from the first measurement by more than the second threshold deviation.

Clause 3. The infusion system of Clause 2, wherein the second measurement is representative of a first distance between the second sensor and a first side of the drive head, and the third measurement is representative of a second distance between the third sensor and a second side of the drive head, opposite the first side.

Clause 4. The infusion system of Clause 2 or Clause 3, wherein the controller being configured to provide the second alert comprises the controller being configured to: indicate a blockage in a path of the plunger or the drive head; facilitate termination of the motor; and prompt a user to confirm removal of the blockage before the motor is restarted.

Clause 5. The infusion system of any one of Clauses 2 through 4, wherein the first sensor comprises an encoder device and the second sensor comprises an optical transceiver pair, and wherein obtaining the second measurement comprises the optical transceiver pair emitting an optical signal and measuring a portion of the optical signal reflected from a portion of the drive head.

Clause 6. The infusion system of any one of Clauses 2 through 5, wherein the controller is configured to, when the second measurement deviates from the first measurement by more than the first threshold deviation: deactivate the first sensor; and activate the third sensor.

Clause 7. The infusion system of any one of Clauses 1 through 6, wherein the controller is configured to, responsive to the second measurement deviating from the first measurement by more than the first threshold deviation: adjust a speed of the motor responsive to the second measurement deviating from the first measurement by more than the first threshold deviation.

Clause 8. The infusion system of Clause 7, wherein the controller is configured to: adjust the speed of the motor by adjusting the speed to zero by terminating operation of the motor.

Clause 9. The infusion system of Clause 8, wherein the controller is further configured to, prior to terminating operation of the motor: compare at least two new measurements obtained from at least two of the first sensor, second sensor and third sensor; and determine that the at least two new measurements deviate from each other by more than a third threshold deviation.

Clause 10. The infusion system of any one of Clauses 1 through 9, wherein the controller is configured to: continuously determine a volume infused based new measurements obtained from the first sensor; and responsive to the second measurement deviating from the first measurement by more than the first threshold deviation, switch determining of the volume infused from being based on new measurements obtained from the first sensor to being based on new measurements obtained from the second sensor.

Clause 11. A method comprising: under control of one or more processing devices, receiving an indication that a syringe was loaded into a receptacle of an infusion device, the syringe comprising a barrel and a plunger; advancing, while the syringe is loaded in the receptacle, a motor-operated drive head of the infusion device to advance the plunger within the barrel; determining, using a first sensor, a first measurement of a location of the drive head; determining, using a second sensor contemporaneously with obtaining the first measurement, a second measurement of the location of the drive head; determining whether the second measurement deviates from the first measurement by more than a first threshold deviation; and providing a first alert after determining that the second measurement deviates from the first measurement by more than the first threshold deviation.

Clause 12. The method of Clause 11, wherein the first measurement is determined based on operation of the motor, the system further comprising: determining, using a third sensor contemporaneously with obtaining the second measurement, a third measurement of the location of the drive head independent of the motor, the third sensor measuring the location of the drive head from a side of the drive head opposite the second sensor; determining whether the third measurement deviates from the second measurement by more than a second threshold deviation; and providing a second alert when the second measurement deviates from the first measurement by more than the second threshold deviation.

Clause 13. The method of Clause 12, wherein the second measurement is a first distance between the second sensor and a first side of the drive head, and the third measurement is a second distance between the third sensor and a second side of the drive head, opposite the first side.

Clause 14. The method of Clause 12 or Clause 13, wherein providing the second alert comprises: indicating a blockage in a path of the plunger or the drive head; facilitating terminating the motor; and prompting a user to confirm removal of the blockage before the motor is restarted.

Clause 15. The method of any one of Clauses 12 through 14, wherein the first sensor comprises an encoder device and the second sensor comprises an optical transceiver pair, and wherein obtaining the second measurement comprises the optical transceiver pair emitting an optical signal and measuring a portion of the optical signal reflected from a portion of the drive head.

Clause 16. The method of any one of Clauses 12 through 15, the method further comprising, when the second measurement deviates from the first measurement by more than the first threshold deviation: deactivating the first sensor; and activating the third sensor.

Clause 17. The method of any one of Clauses 11 through 16, further comprising: adjusting a speed of the motor responsive to the second measurement deviating from the first measurement by more than the first threshold deviation.

Clause 18. The method of Clause 17, further comprising: adjusting the speed of the motor by adjusting the speed to zero by terminating operation of the motor.

Clause 19. The method of Clause 18, further comprising, prior to terminating operation of the motor: comparing at least two new measurements obtained from at least two of the first sensor, second sensor and third sensor; and determining that the at least two new measurements deviate from each other by more than a third threshold deviation.

Clause 20. The infusion system of any one of Clauses 11 through 19, further comprising: continuously determining a volume infused based new measurements obtained from the first sensor; and responsive to the second measurement deviating from the first measurement by more than the first threshold deviation, switching determining of the volume infused from being based on new measurements obtained from the first sensor to being based on new measurements obtained from the second sensor.

Clause 21. A non-transitory machine-readable medium storing instructions thereon that, when executed by an infusion device, cause the infusion device to perform the method of any one of Clauses 11 through 20.

Clause 21. An infusion device configured to perform the method of any one of Clauses 11 through 20.

### Further Consideration

It is understood that the specific order or hierarchy of steps in the processes disclosed is an illustration of example approaches. Based upon design preferences, it is understood that the specific order or hierarchy of steps in the processes may be rearranged. Some of the steps may be performed simultaneously. The accompanying method claims present elements of the various steps in a sample order, and are not meant to be limited to the specific order or hierarchy presented.

The previous description is provided to enable any person skilled in the art to practice the various aspects described herein. The previous description provides various examples of the subject technology, and the subject technology is not limited to these examples. Various modifications to these aspects will be readily apparent to those skilled in the art, and the generic principles defined herein may be applied to other aspects. Thus, the claims are not intended to be limited to the aspects shown herein, but is to be accorded the full scope consistent with the language claims, wherein reference to an element in the singular is not intended to mean “one and only one” unless specifically so stated, but rather “one or more.” Unless specifically stated otherwise, the term “some” refers to one or more. Pronouns in the masculine (e.g., his) include the feminine and neuter gender (e.g., her and its) and vice versa. Headings and subheadings, if any, are used for convenience only and do not limit the invention described herein.

The predicate words “configured to”, “operable to”, and “programmed to” do not imply any particular tangible or intangible modification of a subject, but, rather, are intended to be used interchangeably. For example, a processor configured to monitor and control an operation, or a component may also mean the processor being programmed to monitor and control the operation or the processor being operable to monitor and control the operation. Likewise, a processor configured to execute code can be construed as a processor programmed to execute code or operable to execute code.

The term automatic, as used herein, may include performance by a computer or machine without user intervention; for example, by instructions responsive to a predicate action by the computer or machine or other initiation mechanism. The word “example” is used herein to mean “serving as an example or illustration.” Any aspect or design described herein as “example” is not necessarily to be construed as preferred or advantageous over other aspects or designs.

A phrase such as an “aspect” does not imply that such aspect is essential to the subject technology or that such aspect applies to all configurations of the subject technology. A disclosure relating to an aspect may apply to all configurations, or one or more configurations. An aspect may provide one or more examples. A phrase such as an aspect may refer to one or more aspects and vice versa. A phrase such as an “embodiment” does not imply that such embodiment is essential to the subject technology or that such embodiment applies to all configurations of the subject technology. A disclosure relating to an embodiment may apply to all embodiments, or one or more embodiments. An embodiment may provide one or more examples. A phrase such as an “embodiment” may refer to one or more embodiments and vice versa. A phrase such as a “configuration” does not imply that such configuration is essential to the subject technology or that such configuration applies to all configurations of the subject technology. A disclosure relating to a configuration may apply to all configurations, or one or more configurations. A configuration may provide one or more examples. A phrase such as a “configuration” may refer to one or more configurations and vice versa.

As used herein a “user interface” (also referred to as an interactive user interface, a graphical user interface or a UI) may refer to a network based interface including data fields and/or other control elements for receiving input signals or providing electronic information and/or for providing information to the user in response to any received input signals. Control elements may include dials, buttons, icons, selectable areas, or other perceivable indicia presented via the UI that, when interacted with (e.g., clicked, touched, selected, etc.), initiates an exchange of data for the device presenting the UI. A UI may be implemented in whole or in part using technologies such as hyper-text mark-up language (HTML), FLASH™, JAVA™ .NET™, C, C++, web services, or rich site summary (RSS). In some embodiments, a UI may be included in a stand-alone client (for example, thick client, fat client) configured to communicate (e.g., send or receive data) in accordance with one or more of the aspects described. The communication may be to or from a medical device or server in communication therewith.

As used herein, the terms “determine” or “determining” encompass a wide variety of actions. For example, “determining” may include calculating, computing, processing, deriving, generating, obtaining, looking up (e.g., looking up in a table, a database, or another data structure), ascertaining and the like via a hardware element without user intervention. Also, “determining” may include receiving (e.g., receiving information), accessing (e.g., accessing data in a memory) and the like via a hardware element without user intervention. “Determining” may include resolving, selecting, choosing, establishing, and the like via a hardware element without user intervention.

As used herein, the terms “provide” or “providing” encompass a wide variety of actions. For example, “providing” may include storing a value in a location of a storage device for subsequent retrieval, transmitting a value directly to the recipient via at least one wired or wireless communication medium, transmitting or storing a reference to a value, and the like. “Providing” may also include encoding, decoding, encrypting, decrypting, validating, verifying, and the like via a hardware element.

As used herein, the term “message” encompasses a wide variety of formats for communicating (e.g., transmitting or receiving) information. A message may include a machine readable aggregation of information such as an XML document, fixed field message, comma separated message, JSON, a custom protocol, or the like. A message may, in some implementations, include a signal utilized to transmit one or more representations of the information. While recited in the singular, it will be understood that a message may be composed, transmitted, stored, received, etc. in multiple parts.

As used herein, the term “selectively” or “selective” may encompass a wide variety of actions. For example, a “selective” process may include determining one option from multiple options. A “selective” process may include one or more of: dynamically determined inputs, preconfigured inputs, or user-initiated inputs for making the determination. In some implementations, an n-input switch may be included to provide selective functionality where n is the number of inputs used to make the selection.

As user herein, the terms “correspond” or “corresponding” encompasses a structural, functional, quantitative and/or qualitative correlation or relationship between two or more objects, data sets, information and/or the like, preferably where the correspondence or relationship may be used to translate one or more of the two or more objects, data sets, information and/or the like so to appear to be the same or equal. Correspondence may be assessed using one or more of a threshold, a value range, fuzzy logic, pattern matching, a machine learning assessment model, or combinations thereof.

In any embodiment, data generated or detected can be forwarded to a “remote” device or location, where “remote,” means a location or device other than the location or device at which the program is executed. For example, a remote location could be another location (e.g., office, lab, etc.) in the same city, another location in a different city, another location in a different state, another location in a different country, etc. As such, when one item is indicated as being “remote” from another, what is meant is that the two items can be in the same room but separated, or at least in different rooms or different buildings, and can be at least one mile, ten miles, or at least one hundred miles apart. “Communicating” information references transmitting the data representing that information as electrical signals over a suitable communication channel (e.g., a private or public network). “Forwarding” an item refers to any means of getting that item from one location to the next, whether by physically transporting that item or otherwise (where that is possible) and includes, at least in the case of data, physically transporting a medium carrying the data or communicating the data. Examples of communicating media include radio or infra-red transmission channels as well as a network connection to another computer or networked device, and the internet or including email transmissions and information recorded on websites and the like.

1. An infusion system comprising:
a receptacle for receiving a syringe, the syringe comprising a barrel and a plunger;
a motor-operated drive head for advancing the plunger within the barrel;
a first sensor for determining a location of the drive head while the syringe is received in the receptacle;
a second sensor for determining the location of the drive head independently of the first sensor and the motor; and
a controller configured to:
operate the motor to advance the plunger within the barrel;
determine, using the first sensor, a first measurement of the location of the drive head while the syringe is received in the receptacle;
determine, using the second sensor contemporaneously with obtaining the first measurement, a second measurement of the location of the drive head;
determine that the second measurement deviates from the first measurement by a first threshold deviation; and
provide a first alert after determining that the second measurement deviates from the first measurement by more than the first threshold deviation.

2. The infusion system of claim 1, wherein the first measurement is determined based on operation of the motor, the system further comprising:
a third sensor for measuring the location of the drive head from a side of the drive head opposite the second sensor,
wherein the controller is further configured to:
determine, using the third sensor contemporaneously with obtaining the second measurement, a third measurement of the location of the drive head;
determine whether the third measurement deviates from the second measurement by more than a second threshold deviation; and
provide a second alert when the second measurement deviates from the first measurement by more than the second threshold deviation.

3. The infusion system of claim 2, wherein the second measurement is representative of a first distance between the second sensor and a first side of the drive head, and the third measurement is representative of a second distance between the third sensor and a second side of the drive head, opposite the first side.

4. The infusion system of claim 2, wherein the controller being configured to provide the second alert comprises the controller being configured to:
indicate a blockage in a path of the plunger or the drive head;
facilitate termination of the motor; and
prompt a user to confirm removal of the blockage before the motor is restarted.

5. The infusion system of claim 2,
wherein the first sensor comprises an encoder device and the second sensor comprises an optical transceiver pair, and
wherein obtaining the second measurement comprises the optical transceiver pair emitting an optical signal and measuring a portion of the optical signal reflected from a portion of the drive head.

6. The infusion system of claim 2, wherein the controller is configured to, when the second measurement deviates from the first measurement by more than the first threshold deviation:
deactivate the first sensor; and
activate the third sensor.

7. The infusion system of claim 1, wherein the controller is configured to, responsive to the second measurement deviating from the first measurement by more than the first threshold deviation:
adjust a speed of the motor responsive to the second measurement deviating from the first measurement by more than the first threshold deviation.

8. The infusion system of claim 7, wherein the controller is configured to:
adjust the speed of the motor by adjusting the speed to zero by terminating operation of the motor.

9. The infusion system of claim 8, wherein the controller is further configured to, prior to terminating operation of the motor:
compare at least two new measurements obtained from at least two of the first sensor, second sensor and third sensor; and
determine that the at least two new measurements deviate from each other by more than a third threshold deviation.

10. The infusion system of claim 1, wherein the controller is configured to:
continuously determine a volume infused based new measurements obtained from the first sensor; and
responsive to the second measurement deviating from the first measurement by more than the first threshold deviation, switch determining of the volume infused from being based on new measurements obtained from the first sensor to being based on new measurements obtained from the second sensor.

11. A method comprising:
under control of one or more processing devices,
receiving an indication that a syringe was loaded into a receptacle of an infusion device, the syringe comprising a barrel and a plunger;
advancing, while the syringe is loaded in the receptacle, a motor-operated drive head of the infusion device to advance the plunger within the barrel;
determining, using a first sensor, a first measurement of a location of the drive head;
determining, using a second sensor contemporaneously with obtaining the first measurement, a second measurement of the location of the drive head;
determining whether the second measurement deviates from the first measurement by more than a first threshold deviation; and
providing a first alert after determining that the second measurement deviates from the first measurement by more than the first threshold deviation.

12. The method of claim 11, wherein the first measurement is determined based on operation of the motor, the system further comprising:
determining, using a third sensor contemporaneously with obtaining the second measurement, a third measurement of the location of the drive head independent of the motor, the third sensor measuring the location of the drive head from a side of the drive head opposite the second sensor;
determining whether the third measurement deviates from the second measurement by more than a second threshold deviation; and
providing a second alert when the second measurement deviates from the first measurement by more than the second threshold deviation.

13. The method of claim 12, wherein the second measurement is a first distance between the second sensor and a first side of the drive head, and the third measurement is a second distance between the third sensor and a second side of the drive head, opposite the first side.

14. The method of claim 12, wherein providing the second alert comprises:
indicating a blockage in a path of the plunger or the drive head;
facilitating terminating the motor; and
prompting a user to confirm removal of the blockage before the motor is restarted.

15. The method of claim 12, wherein the first sensor comprises an encoder device and the second sensor comprises an optical transceiver pair, and wherein obtaining the second measurement comprises the optical transceiver pair emitting an optical signal and measuring a portion of the optical signal reflected from a portion of the drive head.

16. The method of claim 12, the method further comprising, when the second measurement deviates from the first measurement by more than the first threshold deviation:
deactivating the first sensor; and
activating the third sensor.

17. The method of any claim 11, further comprising:
adjusting a speed of the motor responsive to the second measurement deviating from the first measurement by more than the first threshold deviation.

18. The method of claim 17, further comprising:
adjusting the speed of the motor by adjusting the speed to zero by terminating operation of the motor.

19. The method of claim 18, further comprising, prior to terminating operation of the motor:
comparing at least two new measurements obtained from at least two of the first sensor, second sensor and third sensor; and
determining that the at least two new measurements deviate from each other by more than a third threshold deviation.

20. The method of claim 11, further comprising:
continuously determining a volume infused based new measurements obtained from the first sensor; and
responsive to the second measurement deviating from the first measurement by more than the first threshold deviation, switching determining of the volume infused from being based on new measurements obtained from the first sensor to being based on new measurements obtained from the second sensor.

21. A non-transitory machine-readable medium storing instructions thereon that, when executed by an infusion device, cause the infusion device to perform the method of claim 11.

22. An infusion device configured to perform the method of claim 11.
